(19) United States
(12) Patent Application Publication
(10) Pub. No.: US 2026/0325011 A1
(43) Pub. Date: Oct. 22, 2026

(54) CLOSED-LOOP, MOTION-BASED, ADAPTIVE MONITORING AND TREATMENT OF NEUROLOGICAL, MUSCULAR, AND SKELETAL CONDITIONS

(71) Applicant: Georgetown University, Washington, DC (US)

(72) Inventors: Gholam Motamedi, Washington, DC (US); Ophir Frieder, Chevy Chase, MD (US)

(21) Appl. No.: 19/718,453

(22) Filed: Jun. 24, 2026

Related U.S. Application Data
(63) Continuation-in-part of application No. 18/159,847, filed on Jan. 26, 2023, now Pat. No. 12,690,782.
Application No. 18/159,847 is a continuation of application No. 16/889,642, filed on Jun. 1, 2020, now Pat. No. 11,589,781.
(60) Provisional application No. 62/855,664, filed on May 31, 2019.
(60) Provisional application No. 62/910,853, filed on Oct. 4, 2019.

(51) Int. Cl. A61B 5/11; A61B 5/00; G06F 40/205

(52) U.S. Cl. CPC A61B 5/112; A61B 5/4842; A61B 5/7267; A61B 5/7275; A61B 5/742; G06F 40/205; A61B 2562/0219; A61B 2562/0247

(57) ABSTRACT

A system for assessing and improving a gait of a user by capturing multi-modal sensor data of the user, calculating gait metrics quantifying a gait of the user during each sensor data window, and using a transformer-based network (e.g., an LLM) to generate instructions (output via a user interface) or neurostimulations (administered by a neural interface) that induce the user to perform physical movements that are clinically optimal for evaluating and improving the gait of the user. The system forms a closed-loop feedback network in which subsequent sensor data of the user performing the induced physical movement are used as a dynamic input to induce subsequent movements, enabling the transformer-based network to dynamically adapt based on real-time motor output, evaluate the efficacy of each induced movement, monitor the longitudinal degradation of the gait of the user, predict the onset of future disease, and/or suggest future treatment options.

### CROSS-REFERENCE TO RELATED APPLICATIONS

This application is a continuation-in-part of U.S. patent application Ser. No. 18/159,847, filed on Jan. 26, 2023, which is a continuation of U.S. patent application Ser. No. 16/889,642, filed Jun. 1, 2020, now U.S. Pat. No. 11,589,781, which claims priority to U.S. Provisional Application No. 62/855,664, filed May 31, 2019, and U.S. Provisional Application No. 62/910,853, filed Oct. 4, 2019, each of which are hereby incorporated by reference in their entirety.

### FEDERAL FUNDING

None

### BACKGROUND

Neurodegenerative diseases, stroke, ataxia, and Multiple Sclerosis (MS) degrade motor functions over time. Current diagnostic methods rely on periodic, subjective clinical evaluations and standardized motor tasks. These intermittent assessments fail to capture continuous, real-world disease progression and fluctuations. Additionally, existing neural stimulation therapies utilize rigid, pre-programmed electrical pulses that do not adapt dynamically to the patient's immediate physiological state or functional needs.

Accordingly, there is a critical need for an intelligent system capable of continuous, objective, motion-based gait assessment, degenerative rate tracking, future escalation prediction, and adaptive treatment.

### SUMMARY

The disclosed system addresses these needs by capturing multimodal sensor data of the user, calculating gait metrics quantifying a gait of the user during each sensor data window, and using a transformer-based network—for example, a generative, decoder-only large language model (LLM) operating as an autonomous agent that autoregressively generates sequence tokens—to generate instructions (output via a user interface) or neurostimulations (administered by a neural interface) that induce the user to perform physical movements that are clinically optimal for evaluating and improving the gait of the user. The system forms a closed-loop feedback network in which subsequent sensor data of the user performing the induced physical movement are used as a dynamic input to induce subsequent movements, enabling the transformer-based network to dynamically adapt based on real-time motor output, evaluate the efficacy of each induced movement, monitor the longitudinal degradation of the gait of the user, predict the onset of future disease, and/or suggest future treatment options.

### BRIEF DESCRIPTION OF THE DRAWINGS

Aspects of exemplary embodiments may be better understood with reference to the accompanying drawings. The components in the drawings are not necessarily to scale; emphasis, instead, is placed upon illustrating the principles of exemplary embodiments.

FIG. 1 is a schematic diagram of an architecture 100 of a gait analysis system 200 according to an exemplary embodiment of the present invention.

FIG. 2 is a block diagram of the gait analysis system 200 according to an exemplary embodiment of the present invention.

FIG. 3 is a graph of exemplary pressure data.

FIG. 4 is another graph of the exemplary pressure data shown in FIG. 3.

FIG. 5 is another graph of the exemplary pressure data shown in FIG. 3.

FIG. 6 is a graph of exemplary training data of a first individual with a normal gait.

FIG. 7 is a graph of exemplary training data of a second individual with a normal gait.

FIG. 8 is a graph of exemplary training data of a third individual with an abnormal gait.

FIG. 9 is a graph of exemplary pressure data of a fourth individual with an abnormal gait.

FIG. 10 is a diagram of a neural network according to an exemplary embodiment of the present invention.

FIG. 11 is a diagram of a recurrent neural network layer according to an exemplary embodiment of the present invention.

FIG. 12 is a graph illustrating exemplary weights applied to each data point in sensor data or training data 240.

FIG. 13 is a graph of sound recordings and acceleration data captured from a healthy volunteer.

FIG. 14 is a graph of sound recordings and acceleration data captured from a patient that has been diagnosed with Parkinson's disease.

FIG. 15 is a graph of sound recordings and acceleration data captured from a patient with a spastic gait after having suffered a stroke.

FIG. 16 is a spectrogram of a sound recording captured from a healthy volunteer.

FIG. 17 is another spectrogram of the sound recording shown in FIG. 16.

FIG. 18A is a diagram illustrating a system for assessing or improving a gait of a user according to exemplary embodiments.

FIG. 18B is a diagram illustrating the system of FIG. 18A according to exemplary embodiments.

FIG. 19A is a flowchart illustrating a process for inducing a user to perform a physical movement according to exemplary embodiments.

FIG. 19B is a flowchart illustrating the process of FIG. 19A according to other exemplary embodiments.

FIG. 19C is a flowchart illustrating the process of FIGS. 19A-19B according to other exemplary embodiments.

FIG. 19D is a flowchart illustrating the process of FIGS. 19A-19C according to other exemplary embodiments.

FIG. 19E is a flowchart illustrating the process of FIGS. 19A-19D according to other exemplary embodiments.

FIG. 19F is a flowchart illustrating the process of FIGS. 19A-19E according to other exemplary embodiments.

FIG. 19G is a flowchart illustrating the process of FIGS. 19A-19F according to other exemplary embodiments.

### DETAILED DESCRIPTION

Reference to the drawings illustrating various views of exemplary embodiments is now made. In the drawings and the description of the drawings herein, certain terminology is used for convenience only and is not to be taken as limiting the embodiments of the present invention. Furthermore, in the drawings and the description below, like numerals indicate like elements throughout.

### RNN-Based Gait Analysis

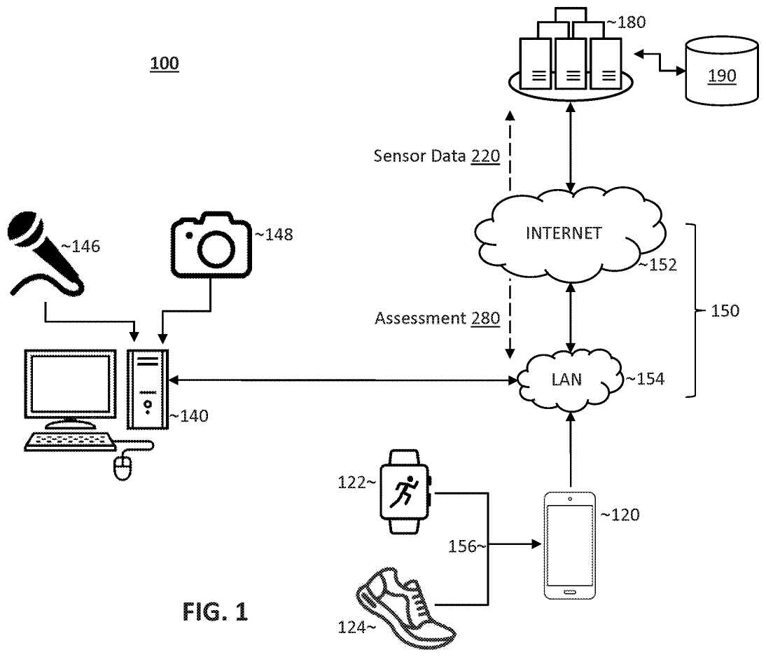

FIG. 1 is a diagram of an architecture 100 of a gait analysis system 200 according to an exemplary embodiment of the present invention.

As shown in FIG. 1, the architecture 100 includes a server 180 that receives sensor data 220 indicative of an individual's gait and outputs an assessment 280 regarding the individual's health. The sensor data 220 may be received via one or more networks 150. The networks 150 may include any combination of the Internet 152, cellular networks, wide area networks (WAN), local area networks (LAN) 154, etc. Communication via the networks 150 may be realized by wired and/or wireless connections.

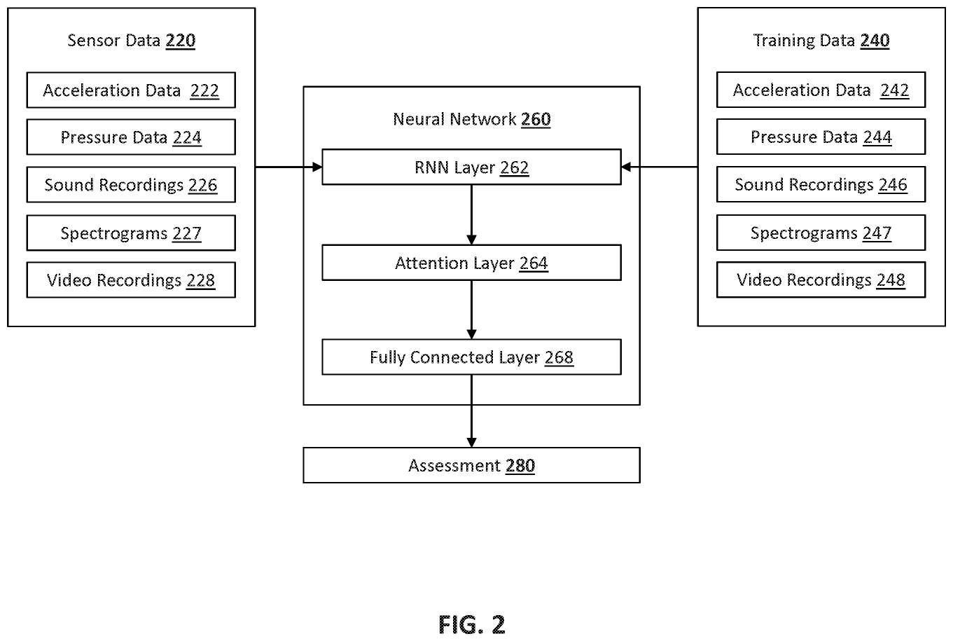

As described in detail below with reference to FIG. 2, sensor data 220 may be captured by a portable electronic device 120 (e.g., a smartphone), a wearable electronic device 122 (e.g., an activity tracker), or sensor-enabled shoes 124. The wearable electronic device 122 or sensor-enabled shoes 124 may communicate with the portable electronic device 120 via direct, short range, wireless communication signals 156 (e.g., Bluetooth). The portable electronic device 120 may transmit sensor data 220 captured by the wearable electronic device 122 or the sensor-enabled shoes 124 to the server 180 via the network(s) 150.

Additionally or alternatively, sensor data 220 may be captured by a microphone 146 or a video camera 148. Sensor data 220 captured by the microphone 146 or video camera 148 may be transmitted to the server 180 by a remote computer 140 via the network(s) 150. The assessment 280 may be output by the server 180 to the remote computer 140 via the network(s) 150.

The server 180 may be any suitable computing device including, for example, an application server or a web server. The server 180 includes non-transitory computer-readable storage media, such as a hard disk or solid-state memory, and at least one hardware computer processor. In addition to internal memory, the server 180 may store data on external (non-transitory) storage media 190, which may be accessed via a wired connection, via a local area network, etc.

The remote computer 140 may be any suitable computing device, such as a desktop or notebook personal computer, configured to output sensor data 220 to the server 180 via the network(s) 150 and receive and display the assessment 280 via a graphical user interface. The remote computer 140 includes non-transitory computer-readable storage media, such as a hard disk or solid-state memory, and at least one hardware computer processor.

The portable electronic device 120 may be any suitable portable computing device including, for example, a smartphone, a tablet computer, a personal digital assistant, etc. The portable electronic device 120 may include a 3-axis accelerometer that measures acceleration in three axes (as described, for example, in U.S. Pat. Pub. No. 2014/0156215 to Eastman, et al.). The portable electronic device 120 may also include a gyroscope that can be used to determine the orientation of the portable electronic device 120 relative to the ground. The accelerometer and the gyroscope (collectively referred to as an inertial measurement unit) may be used by the portable electronic device 120 to measure the acceleration of the portable electronic device 120 relative to the ground in three dimensions. The portable electronic device 120 may also include a microphone that records sound and a camera that records video.

The wearable electronic device 122 may be any suitable wearable computing device including, for example, a fitness tracker, an activity tracker, a smart watch, etc. The wearable electronic device 122 may be configured to be worn by a user, for example, around a wrist, an arm, an ankle, etc. Similar to the portable electronic device 120, the wearable electronic device 122 may include an accelerometer and a gyroscope may be used to determine the acceleration of the wearable electronic device 122 in three dimensions (as described, for example, in U.S. Pat. Pub. No. 2019/0329118 to Balakrishnan, et al.).

The sensor-enabled shoes 124 may be any footwear configured to measure physical parameters as the user walks or runs. The sensor-enabled shoes 124 may be equipped with accelerometers and/or force sensors (as described, for example, in U.S. Pat. Pub. No. 2019/0329118 to Balakrishnan, et al.). The accelerometers may be used to determine the acceleration of each sensor-enabled shoe 124. Additionally or alternatively, the force sensors may be used to determine force applied by each foot.

FIG. 2 is a block diagram of the gait analysis system 200 according to an exemplary embodiment of the present invention.

As shown in FIG. 2, the gait analysis system 200 includes a neural network 260 that receives sensor data 220 and training data 240 and determines and outputs an assessment 280 in response to the sensor data 220. Both the sensor data 220 and the training data 240 may include acceleration data 222 or 242, pressure data 224 or 244, sound recordings 226 or 246, spectrograms 227 or 247, and/or video recordings 228 or 248. The neural network 260 includes a recurrent neural network (RNN) layer 262 and a fully connected layer 268. In a preferred embodiment, the neural network 260 also includes an attention layer 264. As those skilled in the art will recognize, other neural network configurations (e.g., convolutional neural networks (CNN) and transformers) can likewise be used.

The acceleration data 222 (or 242) may be any data indicative of the acceleration of a device carried by a user (e.g., in three axes relative to the ground) over time. The acceleration data 222 (or 242) may be captured, for example, by the portable electronic device 120 or the wearable electronic device 122. In those instances, the acceleration data 222 (or 242) may be indicative of the acceleration of the portable electronic device 120 or the wearable electronic device 122.

The pressure data 224 (or 244) may be any data indicative of the force or pressure applied by each foot of a user over time. The pressure data 224 (or 244) may be captured, for example, by sensor-enabled shoes 124.

The sound recordings 226 (or 246) may be any data indicative of the sound waves created by a user while walking or running over time. The sound recordings 226 (or 246) may be captured, for example, by the portable electronic device 120 or the microphone 146.

The spectrograms 227 (or 247) may be any visual representation of the spectrum of frequencies of the audio signal from the sound recordings 226 (or 246) as the audio signal varies with time.

The video recordings 228 (or 248) may be any image data indicative of a user walking or running over time. The video recordings 228 (or 248) may be captured, for example, by the portable electronic device 120 or the video camera 148.

The training data 240 and the sensor data 220 could theoretically be stored locally, for example by the remote computer 140, if the remote computer 140 was provided with a data storage capability. The remote computer 140 could also theoretically train and execute the neural network 260 if the remote computer 140 was provided with sufficient computing power and/or time. (Alternatively, the sensor data 220 could theoretically be provided directly to the server 180 without the use of any of the network(s) 150.) As a practical matter, however, the server 180 will be required to train and execute the neural network 260, using training data 240 stored in the storage media 190, having been provided with sensor data 220 via the network(s) 150.

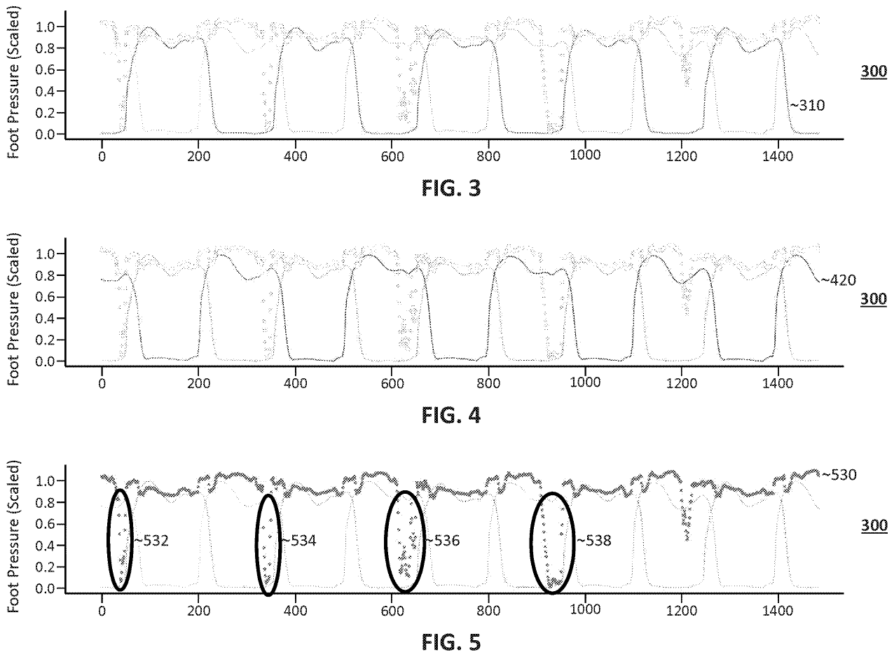

FIGS. 3-5 are a graph 300 of exemplary pressure data 224 or 244. As shown in FIG. 3, the graph 300 includes left foot pressure data 310 that have been normalized within a ten second window to range from 0 to 1. As shown in FIG. 4, the graph 300 also includes right foot pressure data 420 that have been similarly normalized within a ten second window to range from 0 to 1. In each instance, a window is passed over the pressure data 224 or 244 and a ten second moving average is computed and used to normalize the streaming data. Any pressure data 244 that exceeds the [0,1] bounds may be clipped to the maximum or minimum value. As described in detail below, the neural network 260 uses the left foot pressure data 310 and the right foot pressure data 420 to generate assessments 280 of the “normality” of the user gait. In the process, the neural network 260 parses the sensor data 220 into sensor data windows. Meanwhile, the neural network 260 may generate gait window assessments 530 indicative of the “normality” of the user gait during each sensor data window. The gait window assessments 530 determined by the neural network 260 are shown in FIG. 5, including areas 532, 534, 536, and 538, which are discussed below.

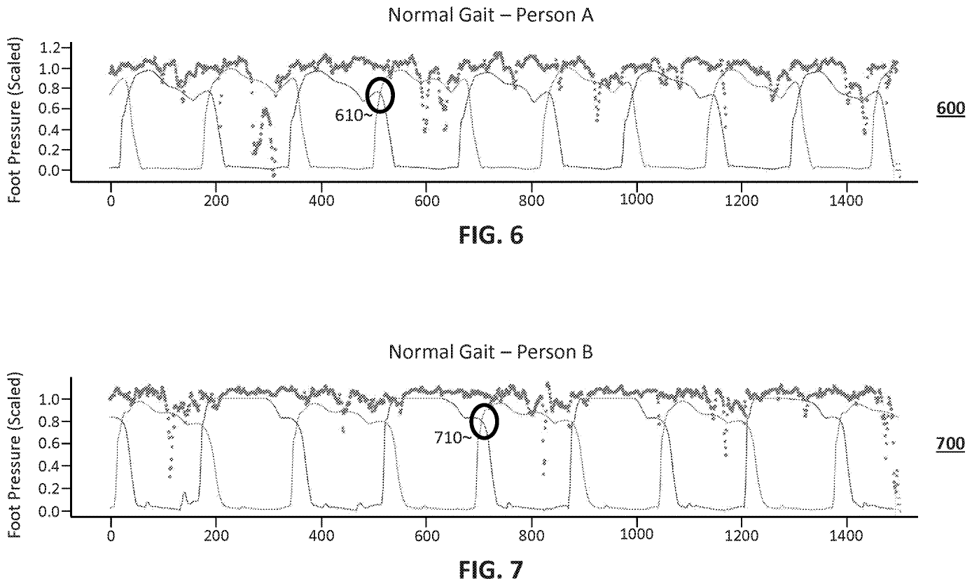

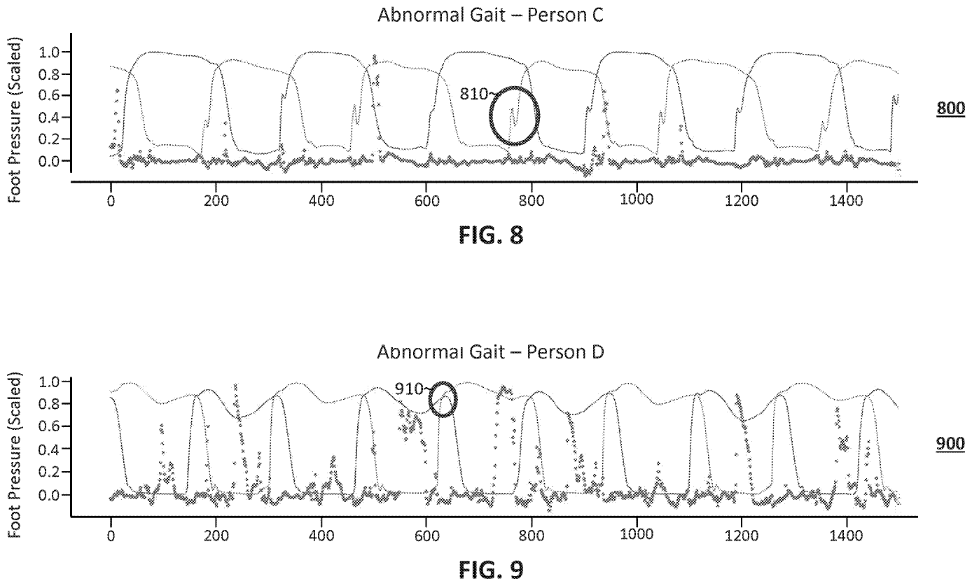

Using training data 240 that include both individuals with diseases that affect gait and individuals with normal gaits, the neural network 260 can be trained to identify diseases by analyzing sensor data 220. FIG. 6 is a graph 600 of exemplary training data 240 (in this instance, pressure data 244) of a first individual with a normal gait. FIG. 7 is a graph 700 of exemplary pressure data 244 of a second individual with a normal gait. No two gaits are exactly the same. However, as shown in area 610 of FIG. 6 and area 710 of FIG. 7, individuals with normal gaits show similar gait features. FIG. 8 is a graph 800 of exemplary pressure data 244 of a third individual with an abnormal gait. FIG. 9 is a graph 900 of exemplary pressure data 244 of a fourth individual with an abnormal gait. As shown in FIGS. 8 and 9, abnormal gaits differ both in progression of disease as well normal variance. Additionally, abnormal features—for example, shown in area 810 of FIG. 8 and area 910 of FIG. 9—are not present in the pressure data 244 of individuals with normal gaits. Patterns that appear in the training data 240 can be used to identify normal and abnormal gait patterns in the sensor data 220. While pressure data 244 are shown in FIGS. 6-9, the neural network 260 can be similarly trained using other kinds of training data 240.

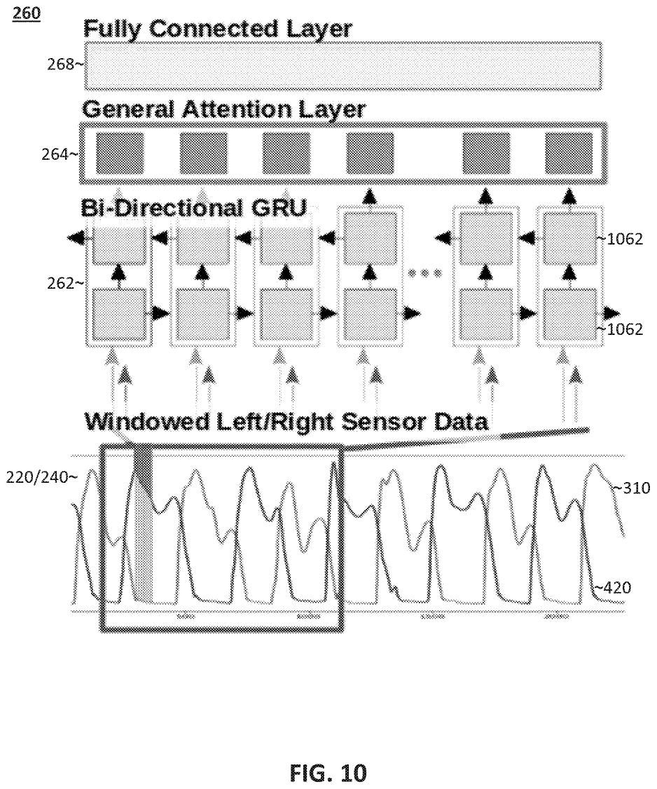

FIG. 10 is a diagram of the neural network 260 according to an exemplary embodiment of the present invention.

As shown in FIG. 10, the training data 240 or the sensor data 220 are provided to the recurrent neural network layer 262. Samples may be grouped into a window and passed to the recurrent neural network layer 262 in series. For example, samples may be grouped into a 1-second window (comprising 30 left foot data points and 30 right foot data points), as preliminary studies have shown that a 1-second window provides optimal results with these gait analysis methods. A sliding window may be used by the recurrent neural network layer 262. However, other windowing methods (e.g., discrete windows) may also be used. A “recurrent neural network layer” is a class of artificial neural networks that takes data of a streaming, sequential, or series nature and provides an output that characterizes the sequence or series. The recurrent neural network begins by reading an input vector and generating an output vector. For all subsequent input vectors provided to the recurrent neural network, the network combines the previous recurrent network output vector and the new input vector to create a new output vector. This process is repeated for all of the input data to create a final characterization of the input stream. By providing the recurrent neural network with multiple sequential or series data points, the recurrent neural network is able to capture both information about individual data points as well as information about the relationships between data points. The recurrent neural network layer 262 shown in FIG. 10 is depicted as a bidirectional recurrent neural network with thirty recurrent neural network nodes 1062 in either direction. In practice, the recurrent neural network 1062 may include a single recurrent neural network node 1062 or a set of multiple recurrent neural network nodes 1062 reading the sensor data 220 or training data 240.

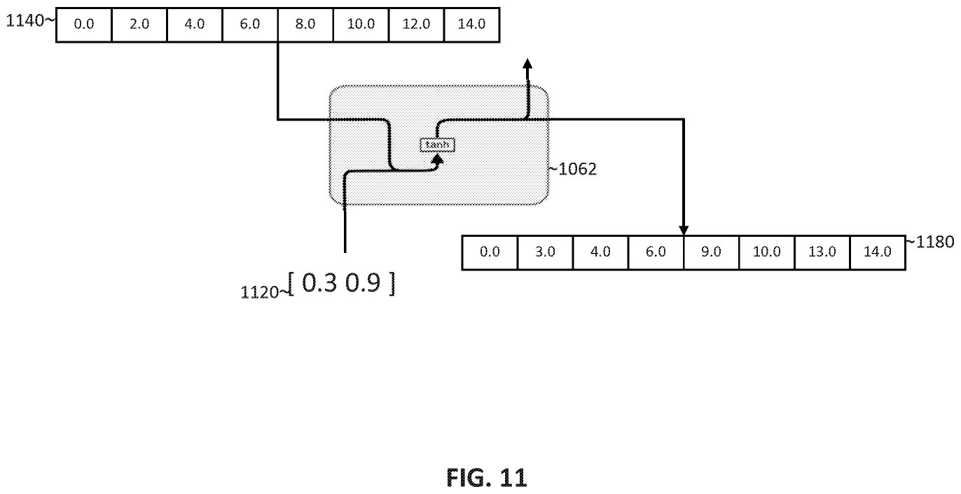

FIG. 11 is a diagram of a recurrent neural network node 1062 of the recurrent neural network layer 262 according to an exemplary embodiment of the present invention.

As each new data point 1120 is received, the recurrent neural network layer 262 creates a context vector 1180 describing the new data point 1120 and the previously determined context vector 1140. As described above, each new data point 1120 may be a sample over time from the sensor data 220 or the training data 240 (in the case of pressure data 224 or 244, for example, a 1-second window comprising 30 left foot data points and 30 right foot data points). The context vector 1180 is a series of numbers that are meaningful only to the neural network 260. However, each context vector 1180 represents a non-human readable intermediate assessment of the normality of the gait of the user for the window, determined based on the new data point 1120 for the window and the previously analyzed data points.

The recurrent neural network layer 262 may be a bidirectional recurrent neural network that creates a first context vector 1180 from a forward pass and second context vector 1180 from a backwards pass. Therefore, the recurrent neural network layer 262 will generate a first context vector 1180 characterizing the new data point 1120 and the sensor data 220 (or the training data 240) that came before the new data point 1120 and a second context vector 1180 characterizing the new data point 1120 and the sensor data 220 (or the training data 240) that came after the new data point 1120. In the preferred embodiment shown in FIG. 11, the recurrent neural network layer 262 is a gated recurrent unit (GRU), a variant of the Long Short Term Memory (LSTM) recurrent neural network (RNN) structure. Internally, the GRU uses an update and reset gate to determine which information should be passed to output. These gates determine how much information from previous data should be saved as well as how the saved data are combined with the incoming data to produce the output. While a GRU is preferred, the recurrent neural network layer 262 may be any recurrent neural network. Meanwhile, to analyze spectrograms 227 and 247, the neural network need not be recurrent.

Referring back to FIG. 10, the context vectors 1180 are provided to the attention layer 264. Not all portions of an individual's gait pattern provide the same amount of information about the normality of that individual's gait. Therefore, different data points in the sensor data 220 have different predictive value for determining whether an individual has an abnormal gait pattern. Accordingly, the attention layer 264 identifies the weight of each data point in the training data 240 and applies those weights to each data point in the sensor data 220.

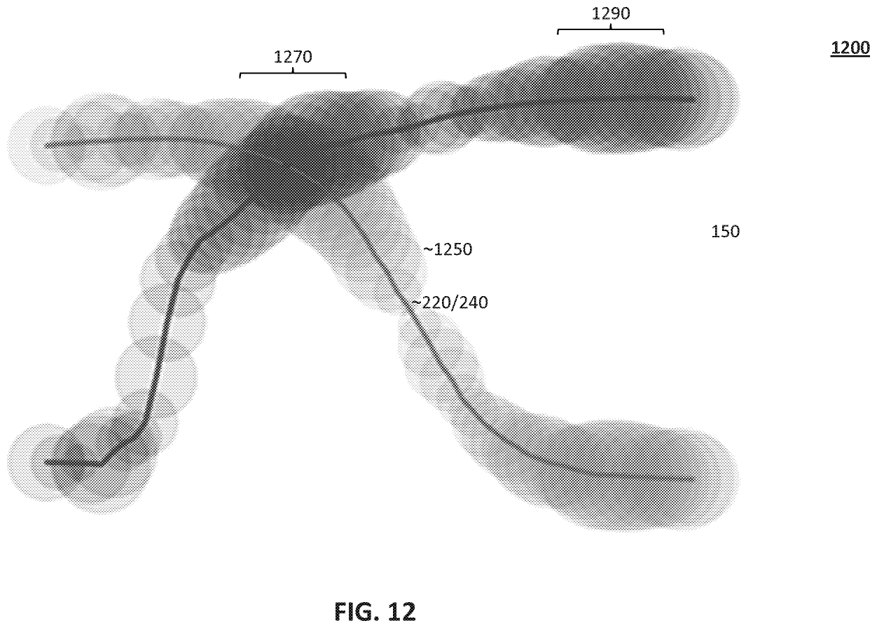

FIG. 12 is a graph 1200 illustrating weights 1250 applied to each data point in the sensor data 220 or the training data 240, where the radius of each circle denotes the amount of attention to be paid to that point in the sensor data 220 or the training data 240. As shown in FIG. 12, the attention layer 264 may place greater weight on the data points during the crossover period 1270 (when the two foot pressures are equal and the balance shifts from one foot to the other) and during the time period 1290 when all of the pressure is on one foot (while the other has been lifted and is being swung forward).

The attention layer 264 is a fully connected layer that, having been trained using the training data 240, takes each context vector 1180 and provides a weight 1250 that represents the importance of that data point. Each context vector 1180 is adjusted by its weight 1250, combined with the other weighted context vectors 1180, and normalized.

Referring back to FIG. 10, the weighted, normalized, and combined context vectors 1180 are provided to the fully connected layer 268.

As one of ordinary skill in the art would recognize, artificial neural networks are computing systems (vaguely inspired by the biological neural networks) that “learn” to perform tasks by considering examples, generally without being programmed with task-specific rules. In this instance, the fully connected layer 268 is trained to recognize normal and abnormal gaits in the sensor data 220 by considering examples of normal and abnormal gaits in the training data 240.

Specifically, the fully connected layer 268 analyzes the context vectors 1180 (generated by the recurrent neural network layer 262) characterizing the sensor data 220 after having been trained on the context vectors 1180 (generated by the recurrent neural network layer 262) characterizing the training data 240. Artificial neural networks are composed of artificial neurons, which receive input and produce output using an output function (developed and refined by the neural network during the network training). The artificial neurons are arranged in layers (e.g., an input layer, one or more optional hidden layers, and an output layer). Connections provide the output of one neuron and the input to another neuron. Each connection has a weight (also developed and refined by the neural network during the network training) that represents the relative importance of that connection. The fully connected layer 268 is “fully connected,” meaning every neuron in one layer has a connection to every neuron in the next layer.

To differentiate between normal and abnormal gaits in the sensor data 220, training data 240 are provided to the neural network 260 that has been coded to indicate whether each piece of training data 240 represents a normal gait or an abnormal gait. Critically, all of the training data 240 captured from a patient that has been diagnosed with a disease that affects the gait are coded as abnormal (even if that patient's gait may appear normal in most instances). The fully connected layer 268 reduces the context vectors 1180 produced by the recurrent neural network layer 262 to an assessment 280 indicating whether the gait of the user gait is normal or abnormal. The assessment 280 may be binary, for example a rating of 1 (indicating a normal gait) or a rating of 0 (indicating an abnormal gait). Alternatively, the fully connected layer 268 may output an assessment 280 representing the probability that the gait of the user is normal or abnormal.

Additionally, the neural network 260 may be trained to identify abnormal gaits indicative of multiple specific diseases. To train the neural network 260 to identify abnormal gaits indicative of multiple specific diseases, training data 240 may be coded to indicate that they were derived from individuals with specific diseases. The neural network 260 is then trained to output an assessment 280 that differentiates between normal and abnormal gaits and distinguishes between the abnormal gaits indicative of different specific diseases.

Additionally, the neural network 260 may be trained to identify abnormal gaits indicative of one or more diseases at differing stages of progression. To train the neural network 260 to identify abnormal gaits indicative of progressive disease(s) at differing stages, training data 240 may be coded to indicate that they were derived from individuals with disease(s) at specific stages of disease progression. The neural network 260 is then trained to output an assessment 280 that differentiates between normal and abnormal gaits and distinguishes between the abnormal gaits indicative of disease(s) at those differing stages of disease progression.

In addition to an assessment 280 of the user's gait based on the sensor data 220 in their entirety, the gait analysis system 200 may generate assessments 530 for each window of the sensor data 220. Referring back to FIG. 5, those gait window assessments 530 may be output to a clinician to provide a granular view of the normality of the user's gait as the user takes individual steps. As shown in FIG. 5, a clinician may view left foot pressure data 310, right foot pressure data 420, and the assessments 530 determined by the gait analysis system 200. In the example shown in FIG. 5, the assessments 530 generated by the gait analysis system 200 indicate instability in areas 532, 534, 536, and 538.

This disclosure describes a neural network 260 with two layers (the recurrent neural network layer 262 and the fully connected layer 268) and an optional attention layer 264. However, as one of ordinary skill in the art will recognize, any of the neural network layers may be a multi-layer neural network. For example, the recurrent neural network layer 262 may use one or more layers wherein the output of the previous recurrent neural network layer is fed to one or more recurrent neural network nodes in the next recurrent neural network layer. Regarding the fully connected layer 268, multi-layer neural networks are able to differentiate between input data with more and more precision. However, because every piece of data characterizing an individual gait is unique, a multi-layer fully connected layer 268 may produce a model that is “overfit” for this particular purpose. In other words, insignificant variations in user gaits may cause a multi-layer neural network to characterize those user gaits as abnormal. Accordingly, in a preferred embodiment, the fully connected layer 268 is a single layer neural network.

The attention layer 264 is an optional feature that reduces the time necessary to train the neural network 260 to distinguish between normal and abnormal gaits. In embodiments without the attention layer 264, the fully connected layer 268 may generate the assessment 280 based on the final context vector 1180, which describes the final data point 1120 and all of the previously analyzed data points (or, if the recurrent neural network layer 262 is bidirectional, a combination of the final context vectors 1180 in either direction). Alternatively, the fully connected layer 268 may generate the assessment 280 based on the final context vector 1180 based on a combination of a number of context vectors 1180. In embodiments that include the attention layer 264, however, the fully connected layer 268 generates the assessment 280 based on all of the context vectors 1180 generated by the recurrent neural network layer 262, as weighted by the attention layer 264.

As described above, the neural network 260 may be trained to analyze sensor data 220 from a user—and generate an assessment 280 characterizing the gait of the user—after having been trained using training data 240 from individuals other than the user. Additionally, if the gait analysis system 200 receives sensor data 220 from the same user over time, the gait analysis system 200 can generate an assessment 280 of the changes to the user gait over time. Accordingly, by analyzing sensor data 220 from the same user over time, the gait analysis system may assess the progress of a progressive disease over time or, conversely, monitor the user as the user recovers (e.g., from a stroke). The gait analysis system 200 can then be used to perform trend analysis and project future demise or recovery. Using those derived forecasts, the gait analysis system 200 can forecast the expected duration, degree, and finality of a change in user gait.

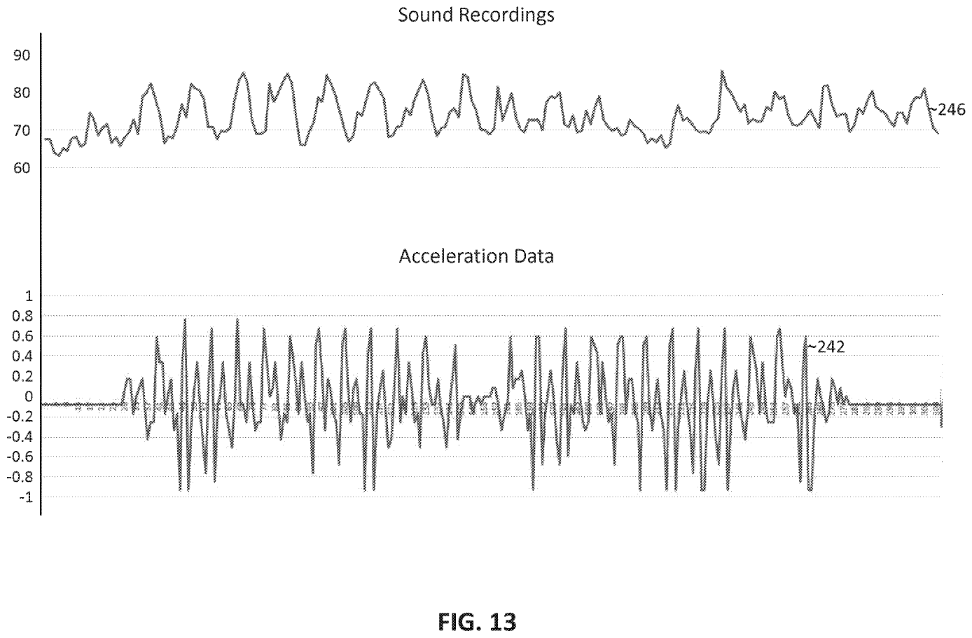

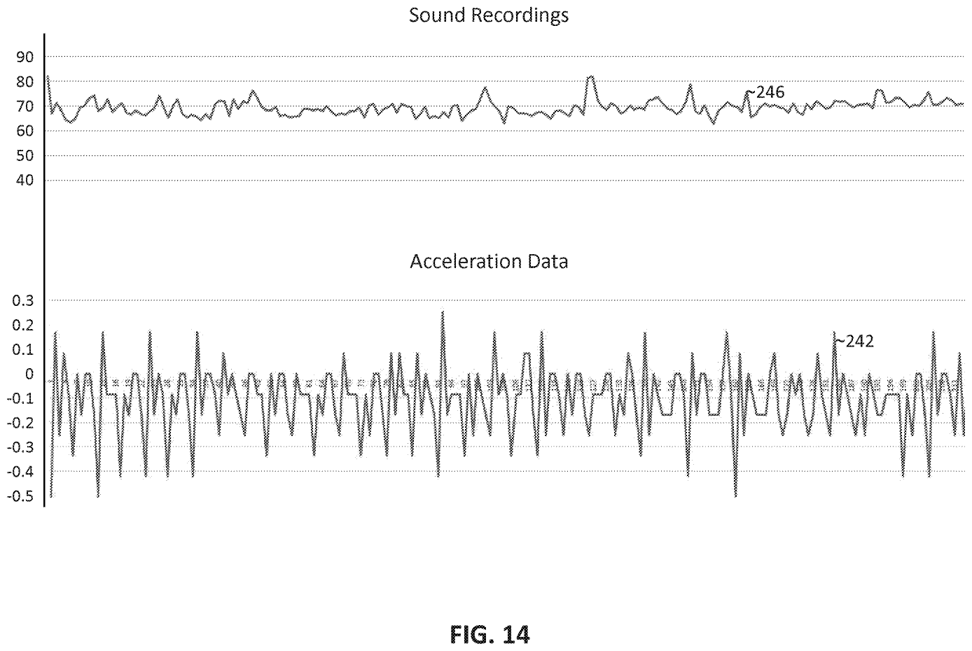

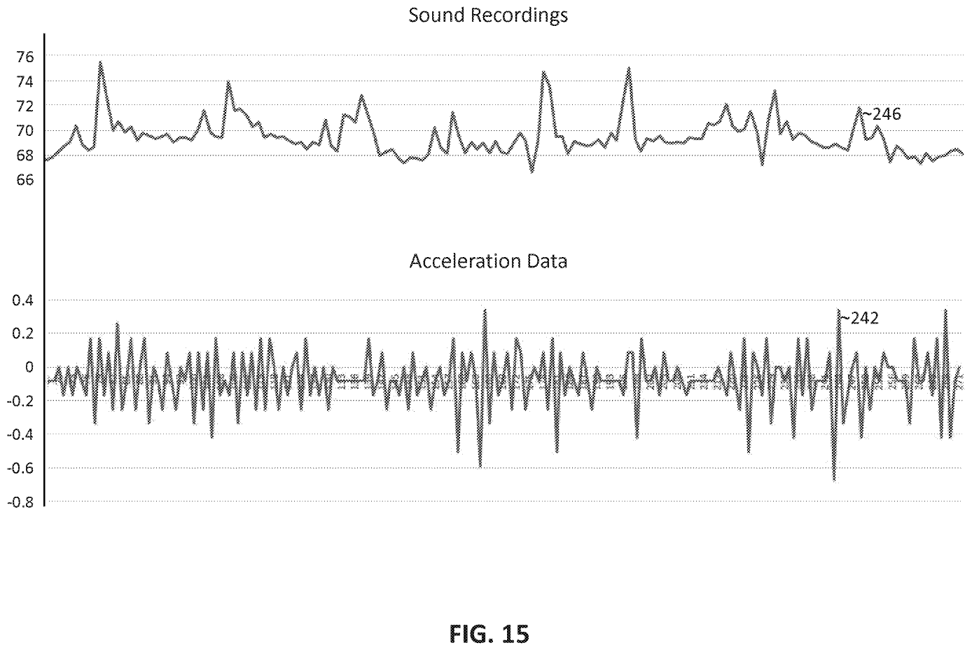

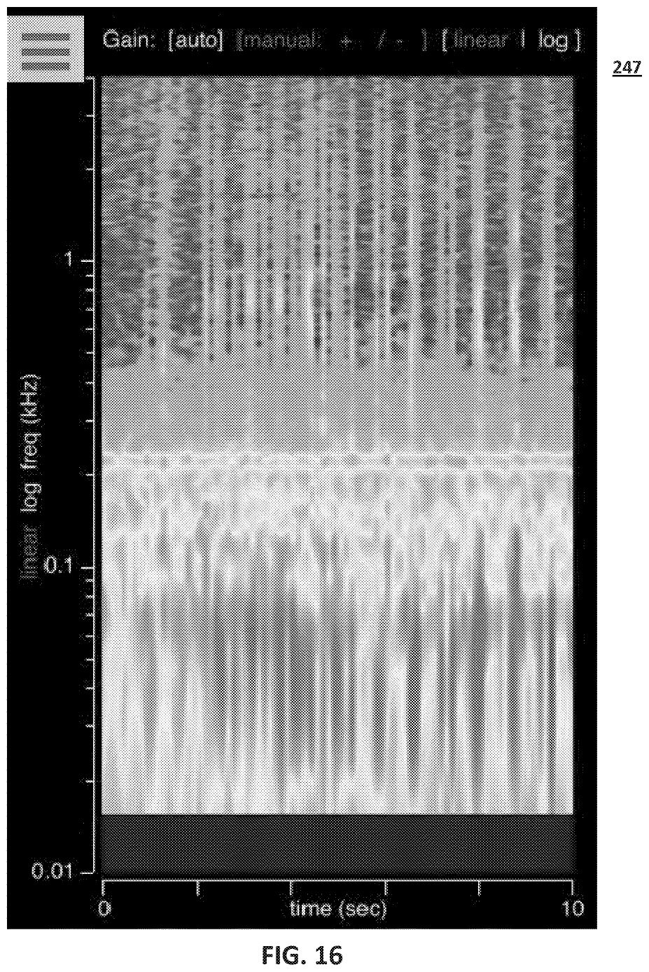

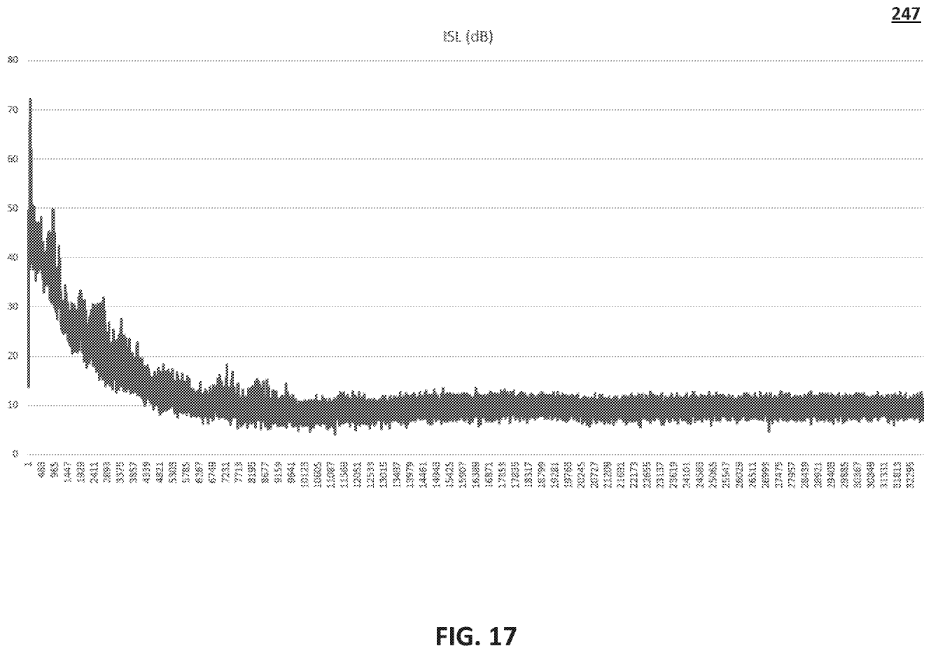

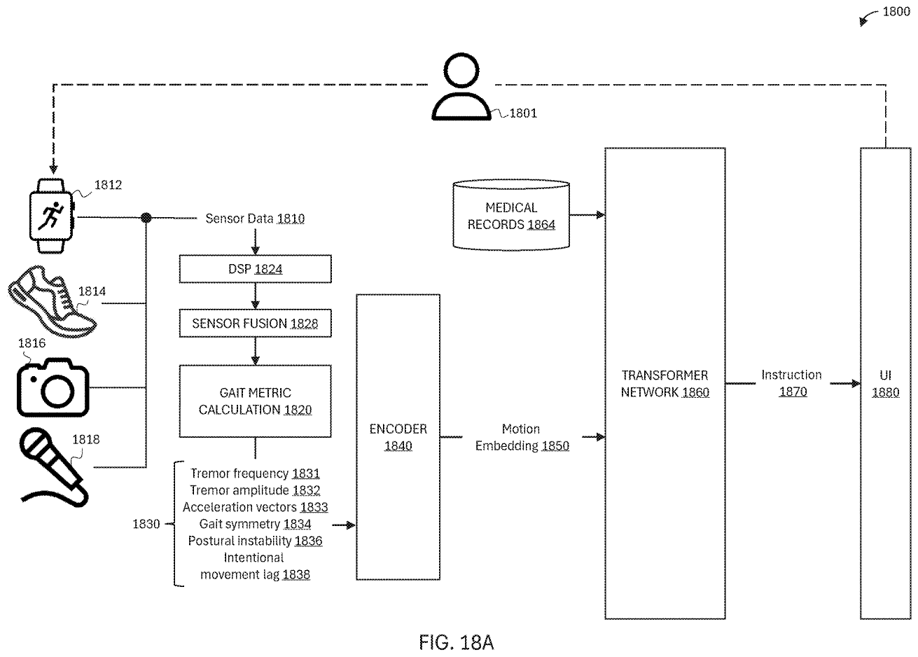

As described above, training data 240 that include pressure data 244 may be used to train the neural network 260 to analyze sensor data 220 that includes pressure data 224. However, the same neural network 260 may be used to analyze other types of sensor data 220 (e.g., acceleration data 222, sound recordings 226, spectrograms 227, video recordings 228, etc.) if provided with the same type training data 240 (e.g., acceleration data 242, sound recordings 246, spectrograms 247, video recordings 248, etc.). FIG. 13 is a graph of sound recordings 246 and acceleration data 242 captured from a healthy volunteer. FIG. 14 is a graph of sound recordings 246 and acceleration data 242 captured from a patient that has been diagnosed with Parkinson's disease. FIG. 15 is a graph of sound recordings 246 and acceleration data 242 captured from a patient with a spastic gait after having suffered a stroke. FIGS. 16 and 17 are spectrograms 247 of sound recordings 246 captured from a healthy volunteer.

In some embodiments, the gait analysis system 200 may be trained with different types of training data 240 (e.g., acceleration data 242, pressure data 244, etc.) in parallel, meaning the neural network 260 may be supplied with different types of training data 240 representing the same user gait. If trained in parallel, the neural network 260 will recognize connections between different types of training data 240. Specifically, the recurrent neural network layer 262 will generate the same context vectors 1180 to characterize the same user gait regardless of whether that user gait was identified by acceleration data 242, pressure data 244, etc. Training the neural network 260 in parallel provides many important technical benefits. As a general matter, an assessment 280 generated while analyzing one type of sensor data 220 (e.g., acceleration data 222) is based on training that occurred while analyzing all types of training data 240 (rather than simply acceleration data 242). As a specific matter, the neural network 260 can analyze sensor data 220 from the same user over time- and generate an assessment 280 of the change in gait characteristics-even if different types of user sensor data 220 are available from different time periods.

As described above, the gait analysis system 200 may be trained to recognize gait characteristics associated with certain diseases, such as Parkinson's disease or parkinsonism, peripheral neuropathy, muscle disorders (e.g., ALS, muscular dystrophy, etc.), cerebellar disorders (e.g., stroke, tumor, and degenerative disorders), mood disorders, etc. By analyzing sensor data 220 from the same user over time, the gait analysis system 200 may be used to track the progression of the degenerative diseases as a user's gait deviates farther from normal over time or monitor a patient's recovery (e.g., after a stroke) as the gait of the user approaches normal.

The gait analysis system 200 may also be trained to recognize injuries that affect the gait characteristics of an individual, such as a fall, an injury to a hamstring or muscle, a concussion, other sport injuries, etc. Specifically, the gait analysis system 200 may receive sensor data 220 from the same user over time (e.g., sensor data 220 taken on a run before a practice and sensor data 220 taken on a run after practice). Having been trained with training data 240 from individuals who suffered injuries affecting their gait, the gait analysis system 200 may then recognize whether the user has suffered such an injury in the intervening time period.

As described above, prior art gait analysis methods identify diseases by analyzing a patient's gait characteristics (e.g., strike distance, foot lift, foot pressure, etc.) over a number of steps. However, those methods may be insufficient to identify gait abnormalities that are gradual, episodic, and/or random (such as “freezing of gait”). By generating context vectors 1180 that represent intermediate assessments of the normality of the user gait, the gait analysis system 200 provides important technical and clinical benefits not provided by prior art gait analysis methods. By assessing window-level sensor data 220, the gait analysis system 200 is able to perform event detection and identify gait abnormalities that are gradual, episodic, and/or random. Additionally, the gait analysis system 200 may generate gait window assessments 530 that provide a granular view of the normality of the user's gait as the user takes individual steps.

### LLM-Based, Closed-Loop, Adaptive Monitoring and Treatment

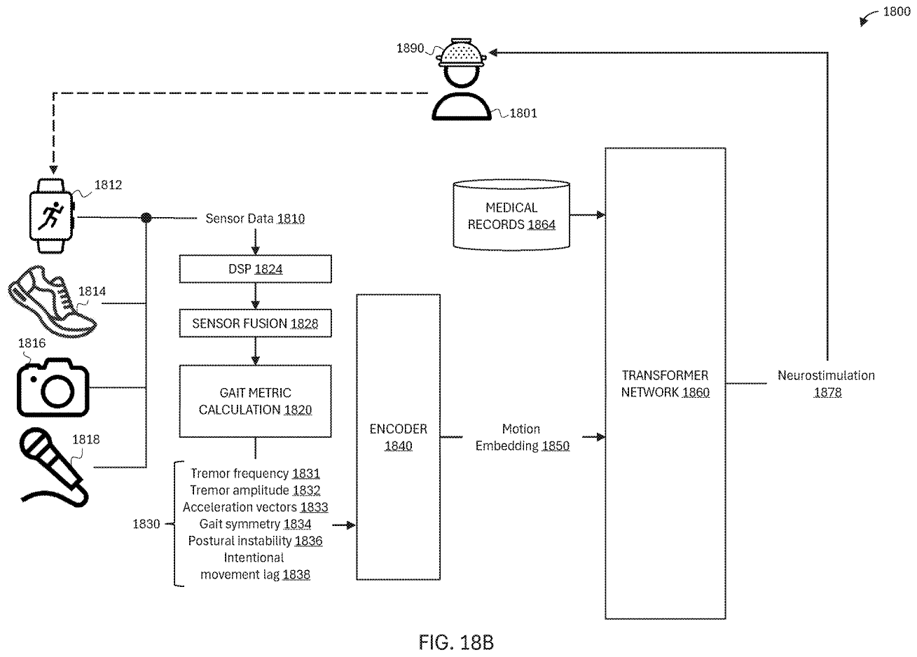

FIGS. 18A and 18B are diagrams of a system 1800 according to exemplary embodiments. As shown in FIGS. 18A and 18B, the system 1800 includes a gait metric calculation module 1820, an encoder 1840, and a transformer-based network 1860.

In the embodiments of FIGS. 18A and 18B, sensor data 1810 are captured from a user 1801 and used, by a gait metric calculation module 1820, to generate gait metrics 1830 quantifying characteristics of the gait of the user 1801. The gait metrics 1830 (e.g., kinematic variables) calculated by the gait metric calculation module 1820 may include any quantifiable physical parameter defining the geometry, velocity, and pattern of motion of the body of the user 1801, independent of the forces that cause that motion. As shown in FIGS. 18A and 18B, for example, the gait metrics 1830 calculated by the gait metric calculation module 1820 may include tremor frequency 1831, tremor amplitude 1832, acceleration vectors 1833, gait symmetry 1834, postural instability 1836, intentional movement lag 1838, etc. As described in more detail below, the sensor data 1810 may be captured continuously (e.g., in real time), and the gait metric calculation module 1820 may generate gait metrics 1830 quantifying characteristics of the user gait during each sensor data window of a predetermined length (e.g., each 1 second sensor data window).

Multi-modal sensor data 1810 may be captured from the user 1801 and used to generate the gait metrics 1830. As shown in FIGS. 18A and 18B, for example, sensor data 1810 may include inertial measurement unit (IMU) data captured by tri-axial accelerometers, gyroscopes, magnetometers (e.g., included in a wearable health monitoring device 1812, a smartphone, etc.), pressure data captured by a wearable sensor 1814, video images captured by a video camera 1816 (e.g., a high-resolution optical tracking camera), audio recordings captured by a microphone 1818, an electroencephalogram (EEG) data captured by an EEG sensor, etc.

Tremor frequency 1831 may be indicative of neurological, muscular, and/or skeletal conditions. A Parkinson's disease tremor, for example, is typically characterized by a frequency of approximately 4-6 Hz, essential tremor by a frequency of approximately 6-12 Hz, and cerebellar tremor by a frequency of approximately 3-5 Hz. Tremor amplitude 1832 is generally lower in Parkinson's disease and essential tremor and higher in cerebellar disorders, though amplitude may vary with disease progression.

In some embodiments, the gait metric calculation module 1820 may calculate tremor frequency 1831 and tremor amplitude 1832 using inertial measurement unit (IMU) data captured by a wearable health monitoring device 1812 (e.g., a tri-axial accelerometer and/or gyroscope worn at the wrist or ankle of the user 1801). To calculate tremor frequency 1831 and tremor amplitude 1832 from IMU data, the gait metric calculation module 1820 may isolate the motion-induced acceleration component by removing the gravitational acceleration component from the raw tri-axial accelerometer signal and apply a band-pass filter to isolate oscillatory motion within a frequency range of clinical interest (e.g., approximately 3-12 Hz, which encompasses the tremor frequencies characteristic of the neurological, muscular, and/or skeletal conditions assessed by the system 1800). The gait metric calculation module 1820 may then apply a frequency-domain transform (e.g., a Fast Fourier Transform (FFT) or power spectral density analysis) to the filtered acceleration signal to identify the dominant frequency peak, which corresponds to the tremor frequency 1831 of the user 1801. The tremor amplitude 1832 may be calculated as the peak-to-peak displacement amplitude or the root-mean-square (RMS) power within the identified frequency band.

In some embodiments, the gait metric calculation module 1820 may calculate tremor frequency 1831 and tremor amplitude 1832 using video images captured by a video camera 1816 (e.g., a high-resolution optical tracking camera or a smartphone camera). To calculate tremor frequency 1831 and tremor amplitude 1832 from video images, the gait metric calculation module 1820 may process each video frame using a pose estimation model (e.g., a convolutional neural network-based pose estimation model such as MediaPipe Pose) that estimates the two-dimensional or three-dimensional coordinates of anatomical landmarks (e.g., joints, fingertips, wrist) of the user 1801 in each frame. The gait metric calculation module 1820 may then construct a time-series trajectory of the coordinates of one or more relevant landmarks (e.g., the fingertips or wrist of the user 1801) across successive frames. To extract tremor frequency 1831 from the landmark trajectory, the gait metric calculation module 1820 may band-pass filter the displacement signal and apply a frequency-domain transform (e.g., FFT or autocorrelation analysis) to identify the dominant oscillatory frequency. The tremor amplitude 1832 may be calculated as the peak-to-peak displacement or RMS displacement of the landmark trajectory within the identified frequency band. In some embodiments, the pixel-based displacement may be converted to metric units (e.g., millimeters) using a calibration reference (e.g., a known physical dimension in the field of view, or an iris-based scaling factor determined by detecting the iris diameter of the user 1801 via facial landmarks).

Additionally, tremor patterns may differ depending on whether the tremor is present at rest or during action (e.g., postural or kinetic tremor), and those patterns may be provided to the transformer-based network 1860 as additional features for inferring the patient state 1920.

Deviations in gait symmetry 1834 are indicative of lateralized neurological, muscular, or skeletal pathology, and the degree of asymmetry may be used by the transformer-based network 1860 as a feature for inferring the patient state 1920. In some embodiments, the gait metric calculation module 1820 may calculate gait symmetry 1834 using IMU data captured by one or more wearable health monitoring devices 1812 positioned bilaterally on the user 1801 (e.g., at the left and right wrist, ankle, or hip). In those embodiments, the gait metric calculation module 1820 may independently calculate temporal gait parameters (e.g., step time, swing time, stance time, and/or double-support time) for each side of the body of the user 1801. Gait symmetry 1834 may then be quantified as a ratio or difference between the bilateral temporal gait parameters (e.g., left step time divided by right step time, or the absolute difference between left and right swing times).

In some embodiments, the gait metric calculation module 1820 may calculate gait symmetry 1834 using pressure data captured by a wearable sensor 1814 (e.g., force sensors integrated into sensor-enabled shoes). In those embodiments, the gait metric calculation module 1820 may compare the force profiles (magnitude and timing) between the left foot and the right foot of the user 1801 to quantify bilateral symmetry in ground reaction forces and temporal gait events.

In some embodiments, the gait metric calculation module 1820 may calculate gait symmetry 1834 using video images captured by a video camera 1816. In those embodiments, the gait metric calculation module 1820 may detect gait events (e.g., initial contact and toe-off) for each limb of the user 1801 from the temporal trajectories of lower-limb anatomical landmarks (e.g., ankle, heel, and toe landmarks) estimated by the pose estimation model, and may calculate bilateral temporal gait parameters from those detected gait events.

In some embodiments, the gait metric calculation module 1820 may calculate gait symmetry 1834 using audio recordings captured by a microphone 1818. As described in the gait analysis system 200 above with reference to FIGS. 1-17, sound recordings of the user 1801 walking are indicative of gait characteristics. The temporal cadence and spectral characteristics of footfall sounds differ by condition. For example, a spastic gait (e.g., following a stroke) may produce an asymmetric auditory pattern characterized by uneven timing between footfalls (e.g., a longer drag followed by a shorter step), a Parkinsonian gait may produce rapid, closely-spaced, low-amplitude footfall sounds characteristic of festination (short, shuffling steps), and a cerebellar ataxic gait may produce widely-spaced, high-amplitude footfall sounds characteristic of a wide-based gait. The gait metric calculation module 1820 may generate a spectrogram (a frequency-domain representation of the audio signal over time) from the audio recordings and analyze the temporal cadence (e.g., the interval between successive footfall events) and spectral characteristics (e.g., the amplitude and frequency content of each footfall) to derive gait symmetry 1834 and/or other gait metrics 1830.

Postural instability 1836 may be indicative of neurological, muscular, and/or skeletal conditions. For example, a double-support time percentage in the range of approximately 20-40% may be clinically normal, with higher percentages indicating postural instability and increased fall risk, particularly in individuals over sixty years of age. In some embodiments, the gait metric calculation module 1820 may calculate postural instability 1836 using IMU data captured by a wearable health monitoring device 1812 positioned at or near the trunk or midline of the user 1801. To calculate postural instability 1836, the gait metric calculation module 1820 may quantify the magnitude and frequency of mediolateral and anteroposterior oscillations (i.e., trunk sway) of the user 1801 during stance or gait. Additionally or alternatively, the gait metric calculation module 1820 may calculate double-support time (i.e., the percentage of time during walking that both feet of the user 1801 are simultaneously in contact with the ground) as an indicator of postural instability 1836 (e.g., a higher double-support time percentage indicating greater reliance on bilateral support to maintain balance).

In some embodiments, the gait metric calculation module 1820 may calculate postural instability 1836 using pressure data captured by a wearable sensor 1814 (e.g., force sensors integrated into sensor-enabled shoes). In those embodiments, the gait metric calculation module 1820 may calculate the center-of-pressure excursion and/or the temporal distribution of force between the left and right feet to quantify postural stability during stance and gait.

Intentional movement lag 1838 refers to the temporal delay between the initiation of a voluntary motor command and the resulting physical movement. Increased intentional movement lag 1838 is clinically indicative of impaired motor planning or slowed neural conduction, as observed in neurodegenerative conditions affecting the basal ganglia or motor cortex pathways. In some embodiments, the system 1800 may receive an electroencephalogram (EEG) of the user 1801 and measure the delay between the neural intention to move and the actual movement captured by the sensor data 1810. In other embodiments, the gait metric calculation module 1820 may measure the delay between an instruction 1870 generated by the transformer-based network 1860 (or a neurostimulation 1878 administered by the neural interface 1890) and the onset of the resulting physical movement detected in the sensor data 1810. For example, the gait metric calculation module 1820 may detect the onset of acceleration in an IMU signal following delivery of an instruction 1870 and calculate the elapsed time between the instruction 1870 and the detected onset.

In some embodiments, the gait metric calculation module 1820 may calculate any of the gait metrics 1830 described above using any combination of the available sensor modalities (e.g., IMU data, pressure data, video images, and/or audio recordings) independently or in combination via the sensor fusion module 1828 as described above.

In some embodiments, a digital signal processing (DSP) module 1824 filters out non-symptomatic noise (e.g., motion artifacts) and normalizes the sensor data 1810. In some embodiments, the gait metric calculation module 1820 independently calculates gait metrics 1830 using sensor data 1810 from each available sensor modality (e.g., IMU data, pressure data, etc.) over each sensor data window. In other embodiments, a sensor fusion module 1828 combines all of the available sensor data 1810 captured during each sensor data window such that gait metric calculation module 1820 calculates a single set of gait metrics 1830 for each sensor data window. In those embodiments, for example, the sensor fusion module 1828 may apply predetermined weights to each sensor data 1810 mode (e.g., IMU data may be given more weight than pressure data, which may be weighted higher than audio recordings.)

As described above, each set of gait metrics 1830 forms a digital representation of the gait of the user 1801 during each sensor data window. To provide those digital representations to a transformer-based network 1860, an encoder 1840 is trained to convert each set of gait metrics 1830 into a motion embedding 1850 compatible with the input vector space of the transformer-based network 1860. To convert each set of gait metrics 1830 into a motion embedding 1850 compatible with the input vector space of the transformer-based network 1860, the encoder 1840 may be trained to perform a tokenization process. As used herein, “tokenization” refers to a process of converting discrete operational commands and/or continuous, multi-modal physical data streams (e.g., each set of gait metrics 1830 for each sensor data window) into uniform, digital numerical identifiers (tokens) that match a structured vocabulary index recognized by the transformer-based network 1860.

The encoder 1840 may be realized as a neural network capable of being trained to generate motion embeddings 1850 that meaningfully represent the gait metrics 1830 to the transformer-based network 1860. To train the encoder 1840, for example, the encoder 1840 may generate motion embeddings 1850 for a large corpus of gait metrics 1830 while the transformer-based network 1860 is prompted to decode each motion embedding 1850 to reconstruct the original set of gait metrics 1830. By minimizing the difference between the gait metrics 1830 reconstructed using each motion embedding 1850 and the original set of gait metrics 1830 used to generate that motion embedding 1850, the encoder 1840 is trained to generate motion embeddings 1850 that meaningfully represent the underlying gait metrics 1830 to the transformer-based network 1860.

In some embodiments, the transformer-based network 1860 may be a pre-trained large language model (LLM). In those embodiments, the transformer-based network 1860 may also be provided with medical records 1864 of the user 1801 (e.g., natural language descriptions such as past assessments, treatment goals, prescribed treatments).

In the embodiment of FIG. 18A, the transformer-based network 1860 is trained to generate instructions 1870 for the user 1801 to perform a physical movement, which are output via a user interface (UI) 1880 for the user 1801 to manually perform each physical movement. In those embodiments, the user interface 1880 may be any hardware device suitably configured to output information to the user (e.g., a display, a speaker, a haptic feedback device, etc.). Meanwhile, embodiments of the system 1800 may be configured to output that information to a user 1801 in any physical condition (including a user 1801 that is older, blind, color-blind, deaf, or otherwise disabled). Accordingly, embodiments of the transformer-based network 1860 may be trained to generate natural language instructions 1870 in any language and in any form (e.g., text for output via a display, generated speech for output via a speaker) and/or signals mapped to natural language instructions 1870 (e.g., for output via a haptic feedback device).

In the embodiment of FIG. 18B, the transformer-based network 1860 is trained to generate neurostimulations 1878 causing the user 1801 to perform a physical movement. In those embodiments, the neurostimulations 1878 generated by the transformer-based network 1860 may be output via a neural interface 1890. The neural interface 1890 may be an external or implanted device. For example, the neural interface 1890 may include a hardware pulse generator having microelectrodes implanted in or adjacent to motor cortex pathways, the basal ganglia (a group of structures located deep within the cerebral hemispheres of the brain), or other deep-brain structures of the user 1801. In those embodiments, the neural interface 1890 may be configured to translate the neurostimulations 1878 generated by the transformer-based network 1860 into electrical, optical, or magnetic stimulation profiles. For instance, the neural interface 1890 may be configured to translate neurostimulations 1878 into one of a predetermined set of stimulation profiles that each induce the user 1801 to perform a physical movement. Meanwhile, in those instances, the transformer-based network 1860 may be trained to induce each physical movement by outputting the neurostimulations 1878 causing the neural interface 1890 to output the stimulation profile inducing that physical movement.

Having output instructions 1870 for the user 1801 to perform physical movements (or neurostimulations 1878 causing the user 1801 to perform physical movements), the disclosed system 1800 then captures sensor data 1810 of the user 1801 performing those physical movements. Accordingly, the disclosed system 1800 forms a “closed-loop feedback network.” As used herein, a “closed-loop feedback network” is an automated architecture that alters the physiological state of the user 1801, which is then immediately measured by sensors and used as a dynamic input parameter to recalculate the next iteration of the generated output. In the embodiment of FIG. 18B, for instance, the automated architecture directly alters the physiological state of the user 1801 by generating physical outputs administered via a neural interface.

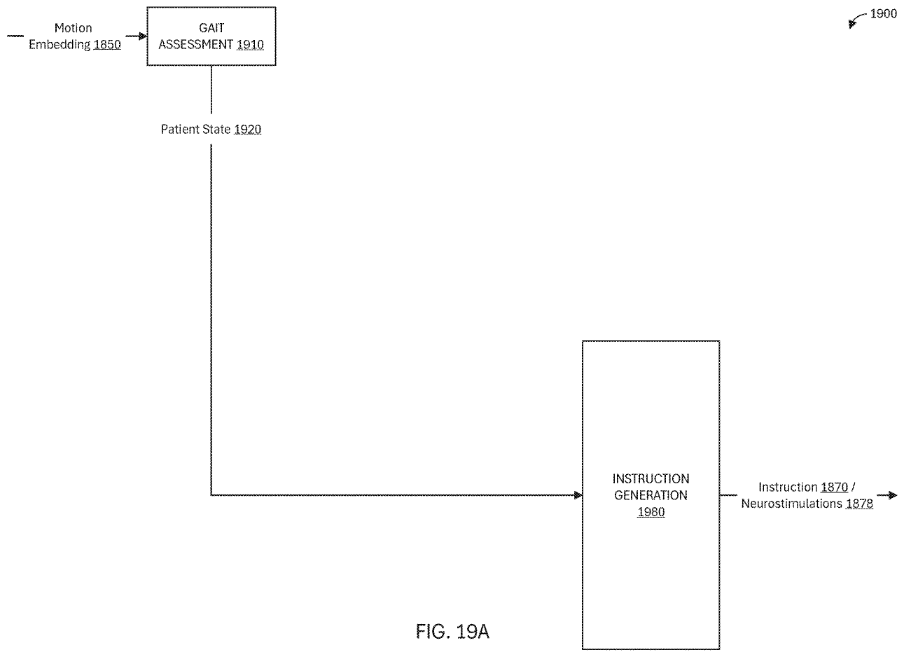

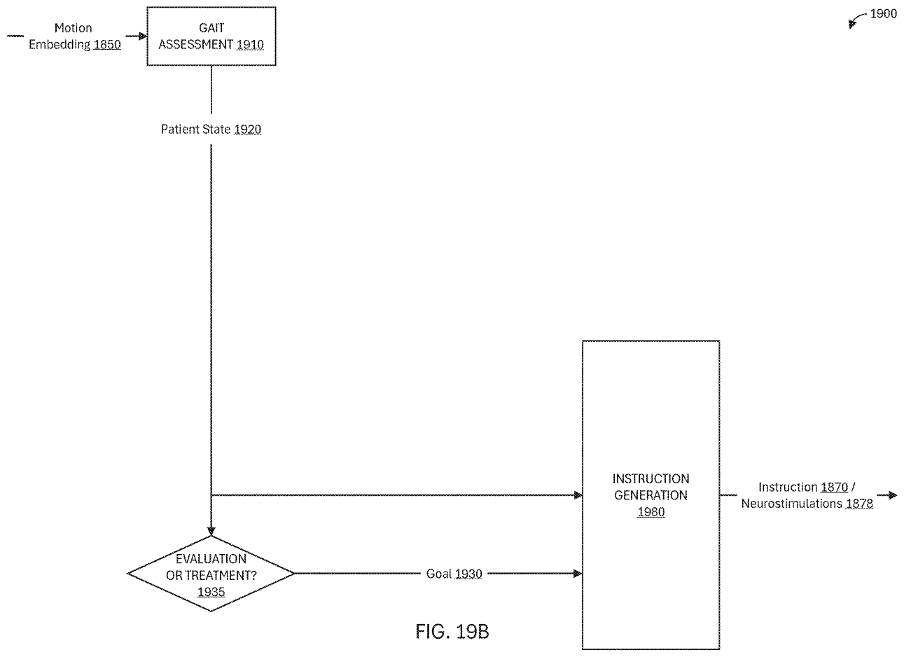

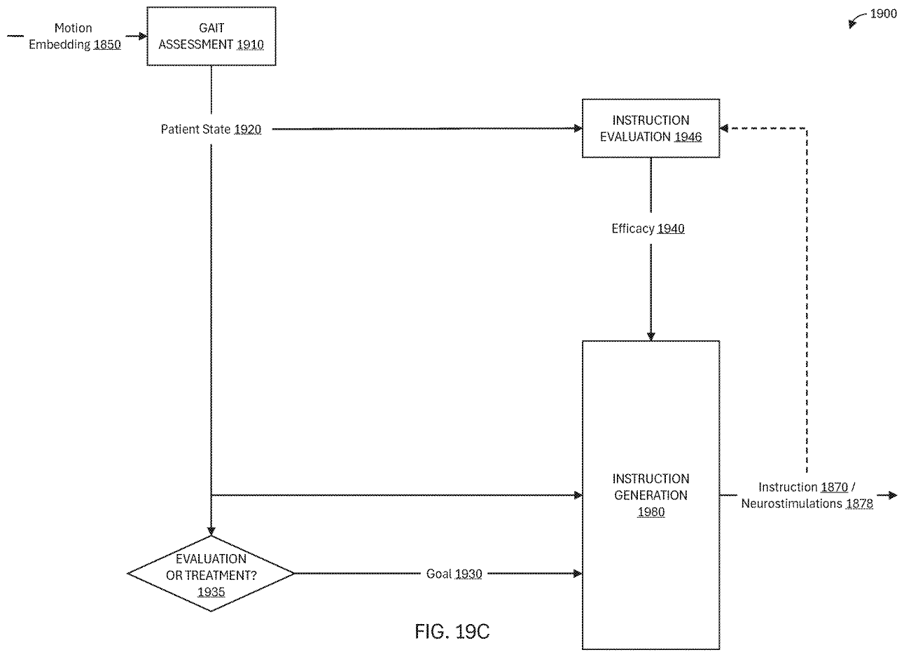

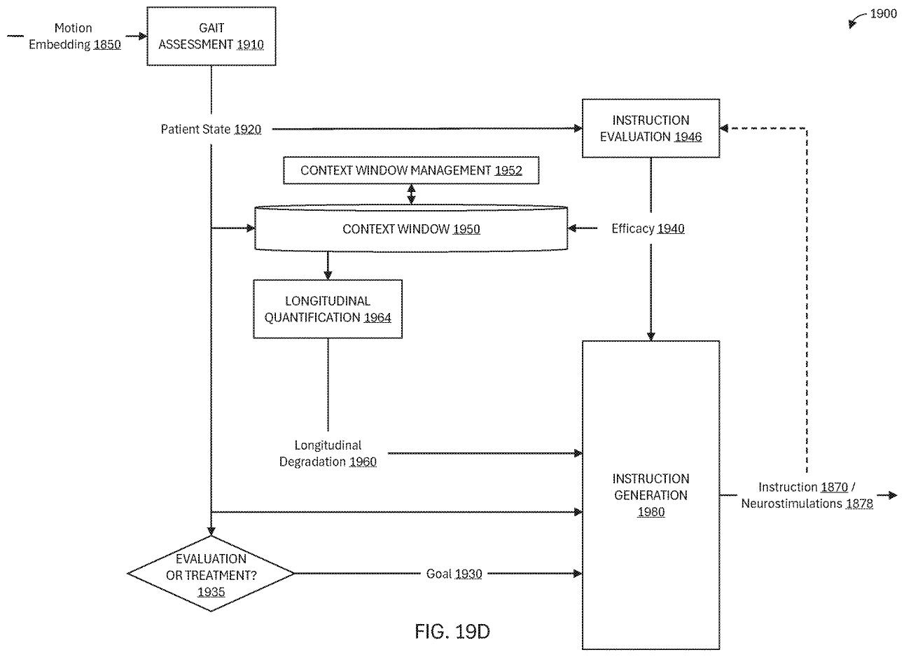

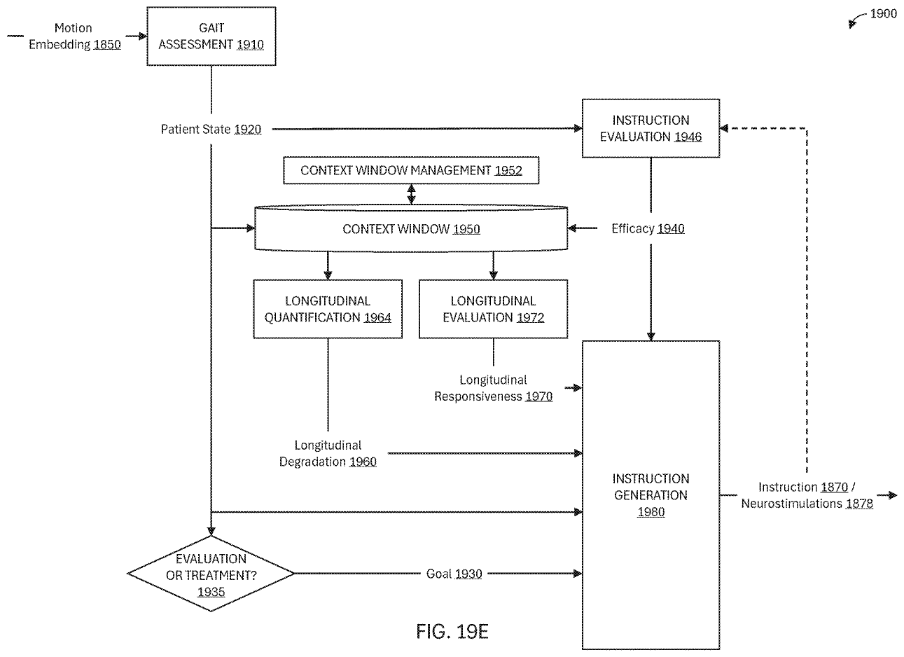

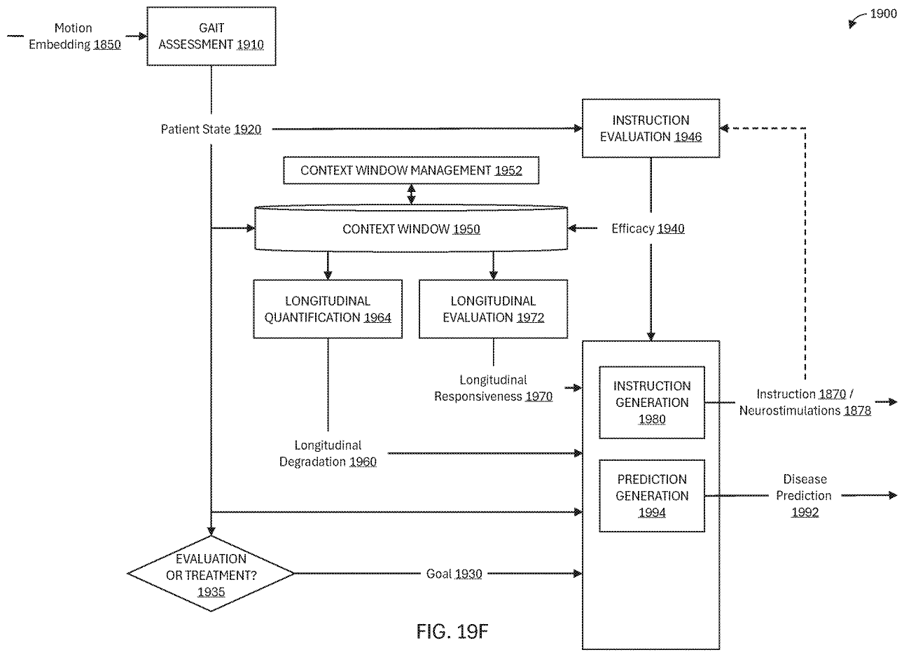

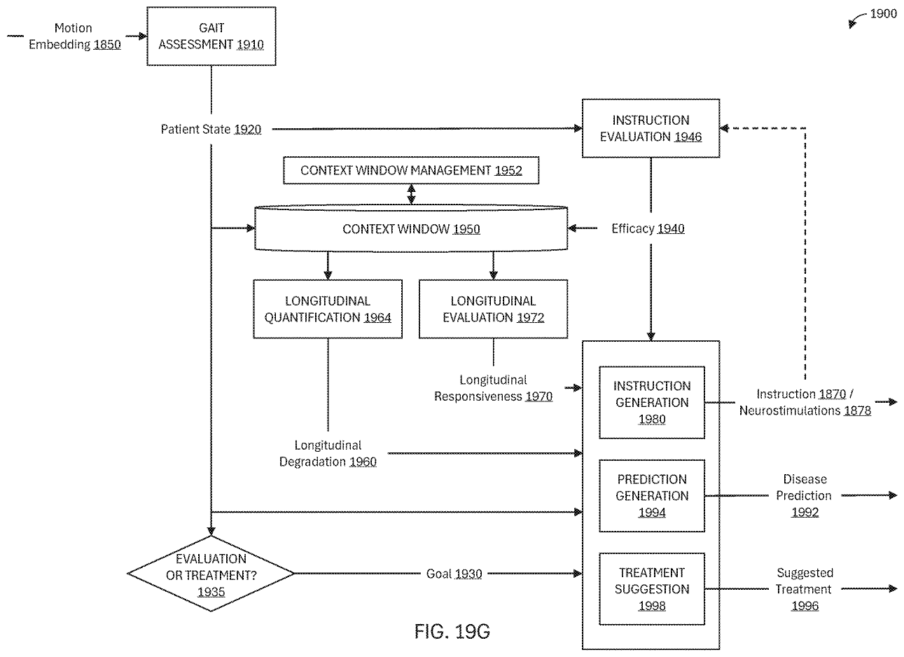

FIGS. 19A-19G are diagrams illustrating a process 1900, executed by the transformer-based network 1860, for generating instructions 1870 (or neurostimulations 1878) according to exemplary embodiments.

In the embodiment of FIG. 19A, the transformer-based network 1860 is trained to infer the patient state 1920 of the user 1801 during each sensor data window based on the motion embedding 1850 generated for that sensor data window (illustrated in FIG. 19A as gait assessment 1910). For example, the transformer-based network 1860 may be trained to detect fine motor changes and/or tremors, classify the gait of the user 1801 as normal or abnormal, classify the gait of the user 1801 as normal or indicative of one of a number of neurological, muscular, and/or skeletal conditions (e.g., Parkinson's, Huntington's Disease, stroke, ataxia, and Multiple Sclerosis, etc.). In other embodiments, rather than deterministic determinations, the transformer-based network 1860 may be trained to generate a probability of fine motor changes, tremors, gait classifications, etc.

In some embodiments, for example, the transformer-based network 1860 may infer the patient state 1920 of the user 1801 by comparing each gait metrics 1830 encoded in the motion embedding 1850 to one or more baselines. Those baselines, for example, may be extracted (by the transformer-based network 1860 or via an external process) from a corpus of medical records, including the medical records of individuals with a normal gait and those of individuals diagnosed with one or more neurological, muscular, and/or skeletal conditions. In some embodiments, for instance, the corpus of medical records may be curated such that the proportion of medical records from individuals with each condition (or with a normal gait) corresponds to the number of individuals with each condition (or with a normal gait) in the population at large.

The transformer-based network 1860 is trained to generate instructions 1870 (or neurostimulations 1878) that are clinically optimal for a user 1801 in the inferred patient state 1920 (illustrated in FIG. 19A as instruction generation 1980). To generate instructions 1870 for the user 1801 to perform physical movements, the transformer-based network 1860 may be trained on a large corpus of patient states 1920 and example instructions 1870 that are clinically optimal for users 1801 in those patient states 1920. To generate neurostimulations 1878, the transformer-based network 1860 may be similarly trained on a large corpus mapping patient states 1920 to clinically optimal physical movements and further trained to select neurostimulations 1878 that induce each predicted physical movement (e.g., one of a predetermined set of stimulation profiles that each induce the user 1801 to perform a physical movement as described above). Alternatively, the transformer-based network 1860 may be trained to predict the clinically optimal physical movements for each patient state 1920 and a separate software module may be configured to algorithmically select the neurostimulations 1878 that induce the physical movements predicted by the transformer-based network 1860.

In some instances, a clinically optimal instruction 1870 or neurostimulation 1878 may be to perform a physical movement that is optimal for improving the gait of a user 1801 in the mapped patient state 1920. In other instances, for example where the transformer-based network 1860 does not have sufficient information to confidently assess the gait of the user 1801, a clinically optimal instruction 1870 or neurostimulation 1878 may be to perform a physical movement that is optimal for assessing the gait of a user 1801 in the mapped patient state 1920. Accordingly, as shown in FIG. 19B, the transformer-based network 1860 may determine whether a goal 1930 of the instruction 1870 or neurostimulation 1878 is to treat the gait of the user 1801 or more accurately evaluate the gait of the user 1801 (illustrated in FIG. 19B as decision 1935) and generate the instruction 1870 or neurostimulation 1878 in view of both the patient state 1920 and the determined goal 1930 (e.g., evaluation or treatment).

As mentioned above, for example, the transformer-based network 1860 may identify the goal 1930 by determining whether the transformer-based network 1860 has sufficient information to confidently assess the gait of the user 1801. If so, the goal 1930 of the instruction 1870 or neurostimulation 1878 may be to perform a physical movement that is optimal for improving the gait of a user 1801 in the mapped patient state 1920. If not, the goal 1930 of the instruction 1870 or neurostimulation 1878 may be to perform a physical movement that is optimal for assessing the gait of a user 1801 in the mapped patient state 1920. In the embodiments of FIG. 19B, for example, the transformer-based network 1860 may be trained on a corpus of example instructions 1870 that are clinically optimal for assessing the gait of users 1801 in a large number of patient states 1920 and example instructions 1870 that are clinically optimal for improving the gait of users 1801 in example patient states 1920.

In the embodiment of FIG. 19C, the transformer-based network 1860 may evaluate the efficacy 1940 of the previously-generated instruction 1870 or neurostimulation 1878 (illustrated in FIG. 19C as instruction evaluation 1946) based on the sensor data 1810 subsequently captured by the user 1801. For example, the transformer-based network 1860 may be trained to evaluate, based on received sensor data 1810 captured in subsequent sensor data windows, whether the user 1801 performed a physical movement included in an instruction 1870 and/or whether a physical movement included in an instruction 1870 (or induced by a neurostimulation 1878) was effective in furthering the determined goal 1930.

In the embodiment of FIG. 19D, each patient state 1920 over time may be stored in the context window 1950 of the transformer-based network 1860. In those embodiments, the transformer-based network 1860 may be trained to assess the longitudinal degradation 1960 or improvement of the gait of the user 1801 over time (illustrated in FIG. 19D as longitudinal quantification 1964) and generate the instruction 1870 or neurostimulation 1878 in view of both the most recent patient state 1920 and the longitudinal degradation 1960 of the user 1801.

In the embodiment of FIG. 19E, the efficacy 1940 of each instruction 1870 or neurostimulation 1878 may be stored in the context window 1950 of the transformer-based network 1860. In those embodiments, the transformer-based network 1860 may be trained to assess the longitudinal responsiveness 1970 of the generated instructions 1870 (and/or neurostimulations 1878) over time (illustrated in FIG. 19E as longitudinal evaluation 1972) and generate the instruction 1870 (or neurostimulation 1878) in view of both the most recent patient state 1920 and the longitudinal responsiveness 1970 of the generated instructions 1870 (and/or neurostimulations 1878) over time. For example, the system 1800 may determine whether physical movements of increasingly higher amplitude or frequency are required to achieve the same efficacy 1940 over time, which is itself a biomarker of disease progression.

Storing each patient state 1920 and the efficacy 1940 of each instruction 1870 over time enables the transformer-based network 1860 to evaluate the longitudinal degradation 1960 of the gait of the user 1801 and/or the longitudinal responsiveness 1970 of the generated instructions 1870 or neurostimulations 1878. Storing all of those data, however, may cause the context window 1950 to store more information than the transformer-based network 1860 can reliably and accurately process (particularly if the system 1800 is used to continuously process sensor data 1810 in real-time). Accordingly, as shown in the FIGS. 19D and 19E, the system 1800 may also include a context window management module 1952 for managing the context window 1950 of the transformer-based network 1860. In those embodiments, the context management module 1952 may summarize or discard past temporal data (e.g., the patient state 1920 from the earliest sensor data windows) to focus on more recent data (e.g., the most recent patient states 1920 and the efficacy 1940 of the most recent instructions 1870 and/or neurostimulations 1878) while maintaining information of ongoing relevance (e.g., the medical records 1864 of the user 1801, the longitudinal degradation 1960 of the gait of the user 1801, the longitudinal responsiveness 1970 of past instructions 1870 and/or neurostimulations 1878).

In some embodiments, the context window management module 1952 may use intermediate layers in the transformer-based network 1860 to identify candidates for summarization or discarding. As described above, the transformer-based network 1860 comprises a plurality of sequential layers, each of which computes attention weights reflecting the relevance of each element stored in the context window 1950 to the current processing step. At each layer, the self-attention mechanism generates a set of attention scores quantifying how strongly the current input attends to each previously-stored element (e.g., each past patient state 1920, each past efficacy 1940 measurement, and/or each past instruction 1870 or neurostimulation 1878) in the context window 1950. To guide the summarization or discarding process, the context window management module 1952 may examine the attention weights computed at one or more intermediate layers of the transformer-based network 1860 during inference. For example, the context window management module 1952 may compute, for each element stored in the context window 1950, a cumulative importance score by aggregating the attention weights assigned to that element across a plurality of attention heads and/or across a plurality of layers. Elements that consistently receive low cumulative attention scores (indicating that their information is no longer being actively retrieved by the transformer-based network 1860 during generation of subsequent instructions 1870 or neurostimulations 1878) may be identified as candidates for summarization or discarding.

Additionally, or alternatively, the context window management module 1952 may analyze the hidden states (i.e., intermediate activations) produced at one or more intermediate layers of the transformer-based network 1860. In a transformer-based architecture, information from earlier tokens in the context window 1950 is progressively absorbed into the hidden state representations at successive layers. Accordingly, the context window management module 1952 may determine that the information encoded by a particular element in the context window 1950 has been sufficiently consolidated into the intermediate hidden states of the transformer-based network 1860 (for example, by determining that removing the element from the context window 1950 does not materially alter the hidden state representations at a predetermined intermediate layer) and may therefore discard that element without material loss of information available to the transformer-based network 1860 during subsequent inference.

In the embodiments described above, the context window management module 1952 may selectively retain elements that continue to receive high attention scores (e.g., the medical records 1864 of the user 1801, recent patient states 1920, and/or baseline measurements used by the transformer-based network 1860 for comparison during gait assessment 1910) while summarizing or discarding elements whose informational contribution has been fully absorbed into the intermediate representations of the transformer-based network 1860 (e.g., patient states 1920 from temporally distant sensor data windows whose trends have already been captured in the longitudinal degradation 1960 or longitudinal responsiveness 1970 inferred by the transformer-based network 1860).

In addition to instructions 1870 (and/or neurostimulations 1878) for inducing the user 1801 to promptly perform physical movements, the system 1800 may enable a clinician to monitor the physiological state 1920 (and/or longitudinal degradation 1960) of the user 1801 and/or the efficacy 1940 (and/or longitudinal responsiveness 1970) of the generated instructions 1870 and/or neurostimulations 1878. For example, the system 1800 may enable a clinician to remotely monitor the user 1801 via one or more networks 150 (see FIG. 1).

In the embodiment of FIG. 19F, the transformer-based network 1860 may be trained to predict the onset of a future disease of the user 1801 (illustrated in FIG. 19F as disease prediction 1992) based on the patient state 1920 and/or efficacy 1940 of each instruction 1870 (and/or neurostimulation 1878) over time (illustrated in FIG. 19F as prediction generation 1994). In those embodiments, for example, the transformer-based network 1860 may be trained on a corpus of medical records 1864 that include qualitative descriptions or quantitative metrics characterizing the gaits of patients that later developed neurological, muscular, and/or skeletal conditions.

In the embodiment of FIG. 19G, the transformer-based network 1860 may be trained to suggest treatment options (illustrated in FIG. 19G as suggested treatment 1996) for the user 1801 (illustrated in FIG. 19G as treatment suggestion 1998). In those embodiments, the transformer-based network 1860 may be similarly trained on a corpus of medical records 1864 of patients diagnosed with developed neurological, muscular, and/or skeletal conditions that specify treatment options suggested for each of those diagnosed conditions. Accordingly, by compiling longitudinal data and generating predictive health trajectories, the disclosed system 1800 enables clinicians to remotely monitor objective biomarkers of degeneration.

In some embodiments, the neurostimulations 1878 generated by the system 1800 may be personalized for the user 1801. For instance, the neurostimulations 1878 generated by the system 1800 may be modified based on limiting physical or mental characteristics of the user 1801 stored by the system 1800. Such characteristics may include, for example, the age of the user 1801, pre-existing comorbidities (e.g., epilepsy, cardiac arrhythmias, or implanted medical devices), cognitive impairments affecting the ability of the user 1801 to comply with generated instructions 1870, medication regimens that alter neural excitability thresholds, prior adverse reactions to stimulation, anatomical variations in electrode placement, or the current stage of disease progression of the user 1801. The transformer-based network 1860 may access these characteristics from the medical records 1864 of the user 1801 and incorporate them as constraints when generating neurostimulations 1878. For example, the transformer-based network 1860 may limit the maximum intensity, frequency, duration, or spatial extent of the generated neurostimulations 1878 to remain within a safe therapeutic window determined for the user 1801 based on the stored characteristics. Additionally or alternatively, the transformer-based network 1860 may select stimulation profiles from a reduced set of predetermined stimulation profiles pre-approved or pre-screened for safety given the particular physical or mental characteristics of the user 1801. As described above, the system 1800 operates as a closed-loop feedback network, and the context window 1950 of the transformer-based network 1860 may store past stimulation parameters and the corresponding efficacy 1940 and any detected adverse effects, such that the personalization of neurostimulations 1878 adapts over time as additional information about the user 1801 is captured by the system 1800.

In addition to the gait assessment 1910 and the instruction generation 1980, the transformer-based network 1860 may perform any combination of the decision 1935, the instruction evaluation 1946, the longitudinal quantification 1964, the longitudinal evaluation 1972, the prediction generation 1994, and/or the treatment suggestion 1998. In some embodiments, the transformer-based network 1860 may be a multi-agent system in which separate agents are trained to separately perform the disclosed processes (e.g., the gait assessment 1910 and the instruction generation 1980). In some embodiments, a single transformer-based network 1860 (e.g., LLM) may be trained to perform the entire process 1900 for generating instructions 1870 (or neurostimulations 1878).

In the gait analysis system 200 described above with reference to FIGS. 1-17, each context vector 1180 generated for each sensor data window represents an interim assessment of the normality of the gait of the user. Similarly, as the process 1900 is executed in real-time, the disclosed system 1800 maintains and continuously updates a working hypothesis of the condition of the user 1801. For example, in the system 1800, the inferred internal layers within the transformer-based network 1860 represent transitory interim assessments of the normality of the gait of the user 1801, the current patient state 1920 and/or longitudinal degradation 1960 of the user 1801, the efficacy 1940 (and/or longitudinal responsiveness 1970) of past instructions 1870 and/or neurostimulations 1878, and/or any disease prediction 1992 or suggested treatment 1996 generated by the transformer-based network 1860 in view of those longitudinal data.

Referring back to FIGS. 18A and 18B, the transformer-based network 1860 may be any network capable of receiving each motion embedding 1850 for each sensor data window as input context and autoregressively generating sequence tokens indicative of the instructions 1870 and/or neurostimulations 1878 described above. For example, the transformer-based network 1860 may be a pre-trained LLM. In some embodiments, the transformer-based network 1860 may have a decoder-only architecture (similar to OpenAI's GPT series, Meta's Llama, etc.). Unlike encoder-decoder structures (e.g., Google's T5, FLAN-T4, Meta's BART), a decoder-only architecture optimizes autoregressive token generation, allowing the model to predict subsequent neural activation tokens (e.g., instructions 1870 and/or neurostimulations 1878) based entirely on historical stimulation sequences (e.g., the efficacy 1940 and/or longitudinal responsiveness 1970 of previous instructions 1870 and/or neurostimulations 1878) and real-time sensory inputs (e.g., gait metrics 1830 calculated based on sensor data 1810 and encoded as motion embeddings 1850).

In some embodiments, the transformer-based network 1860 may be an “agentic” model. As used herein, an “agentic” model is an artificial intelligence model or system architecture configured with the autonomous capacity to evaluate the gait metrics 1830 calculated based on the received sensor data 1810, formulate multi-step execution plans, modify its own generation parameters, and generate the instructions 1870 output via the user interface 1880 (or the neurostimulations 1878 output via the hardware pulse generator 1890) without requiring external or manual human-in-the-loop intervention. For example, the system 1800 may autonomously iterate to modify the frequency, intensity, or spatial targeting of the physical movement induced by the instructions 1870 or neurostimulations 1878.

The system 1800 may be realized as a local device having a local edge processor and local memory (non-transitory computer readable storage media). For example, the local device may be a wearable health monitoring device 1812 having an inertial measurement unit (and/or a microphone 1818) that captures sensor data 1810. Additionally, or alternatively, the local device may receive force data from a wearable sensor 1814 and/or video images captured by a video camera 1816. The local device may include the user interface 1880 and/or neural interface 1890 or may communicate with an external user interface 1880 and/or neural interface 1890. In those embodiments, the local edge processor may perform the digital signal processing 1824, the sensor fusion 1828, and the gait metric calculation module 1820. Alternatively, the sensor data 1810 may be output to a remote server (e.g., via the Internet) that performs those functions. The encoder 1840 may be realized as a local model stored in local memory and executed by the local edge processor. Alternatively, the gait metrics 1830 may be output to a remote server (e.g., via the Internet) that includes the encoder 1840. Finally, the transformer-based network 1860 may be realized as a local model stored in local memory and executed by the local edge processor. Accordingly, the local device may provide instructions 1870 and/or neurostimulations 1878 in real time. Alternatively, the motion embeddings 1850 may be output to a remote server (e.g., via the Internet) that includes the transformer-based network 1860.

While preferred embodiments have been described above, those skilled in the art who have reviewed the present disclosure will readily appreciate that other embodiments can be realized within the scope of the invention. Accordingly, the present invention should be construed as limited only by any appended claims.

What is claimed is:

1. A system, comprising:
non-transitory computer readable storage media that stores sensor data of a user captured by a multi-modal sensor array;
a hardware computer processor that, for each sensor data window of a predetermined length, calculates a set of gait metrics quantifying a gait of the user based on the sensor data captured during the sensor data window;
an encoder that converts each set of gait metrics into a motion embedding for the sensor data window; and
a transformer-based network trained to infer a patient state of the user during each sensor data window based on the motion embedding generated for the sensor data window and generate instructions or neurostimulations inducing the user to perform a physical movement,
wherein the system forms a closed-loop feedback network in which subsequent sensor data of the user performing the induced physical movement are used as a dynamic input to generate subsequent instructions or neurostimulations.

2. The system of claim 1, wherein the transformer-based network uses autoregressive token generation to infer the patient state of the user and induce the user to perform a physical movement that is clinically optimal for the inferred patient state of the user.

3. The system of claim 1, wherein the transformer-based network is further trained to:
determine whether the transformer-based network has sufficient information to infer the patient state of the user; and
select, based on the determination, whether to induce a physical movement that is clinically optimal for evaluating or improving the gait of the user.

4. The system of claim 1, wherein the transformer-based network has a decoder-only architecture.

5. The system of claim 1, wherein the transformer-based network is an agentic model configured to autonomously evaluate each set of gait metrics, formulate multi-step execution plans, modify its own generation parameters, and induce physical movements without requiring external or manual human-in-the-loop intervention.

6. The system of claim 1, wherein the transformer-based network is further trained to:
determine an efficacy of the induced physical movement for each sensor data window based on sensor data captured from the user during a subsequent sensor data window; and
generate instructions or neurostimulations for the subsequent sensor data window based on the patient state of the user and the efficacy of the induced physical movement for the sensor data window.

7. The system of claim 1, wherein:
patient states of the user, each inferred for a respective sensor data window of a plurality of sensor data windows, are stored in a context window of the transformer-based network; and
the transformer-based network is further trained to assess a longitudinal degradation of the gait of the user based at least in part on the patient states of the user inferred for the plurality of sensor data windows.

8. The system of claim 7, further comprising:
a context window management module that summarizes or discards past temporal data stored in the context window while maintaining information of ongoing relevance for inferring a patient state of the user.

9. The system of claim 8, wherein the context window management module identifies past temporal data for summarization or discarding based on intermediate layers of the transformer-based network.

10. The system of claim 8, wherein the transformer-based network is further trained to predict an onset of a future disease of the user based at least in part on the patient states of the user inferred for the plurality of sensor data windows.

11. The system of claim 8, wherein the transformer-based network is further trained to suggest treatment options for the user based at least in part on the patient states of the user inferred for the plurality of sensor data windows.

12. The system of claim 1, further comprising a neural interface configured to translate the neurostimulations generated by the transformer-based network into electrical, optical, or magnetic stimulation profiles.

13. The system of claim 12, wherein the neural interface comprises a hardware pulse generator having microelectrodes implanted in or adjacent to motor cortex pathways, basal ganglia, or other deep-brain structures of the user.

14. The system of claim 1, wherein the encoder is trained to convert each set of gait metrics into a motion embedding compatible with an input vector space of the transformer-based network.

15. The system of claim 1, wherein the encoder performs a tokenization process that converts the sets of gait metrics into uniform, digital numerical identifiers that match a structured vocabulary index recognized by the transformer-based network.

16. The system of claim 1, wherein the sensor data comprise at least one of inertial measurement unit data, pressure data captured by a wearable sensor, video images captured by a video camera, or audio recordings captured by a microphone.

17. The system of claim 1, wherein each set of gait metrics comprises at least one of tremor frequency, tremor amplitude, acceleration vectors, gait symmetry, postural instability, or intentional movement lag.

18. The system of claim 1, further comprising a digital signal processing module configured to filter out non-symptomatic noise and normalize the sensor data.

19. The system of claim 1, wherein the hardware computer processor is configured to:
combine the sensor data captured by the multi-modal sensor array during each sensor data window; and
calculate a single set of gait metrics for each sensor data window based on the combined sensor data captured during the sensor data window.

20. A method of assessing or improving a gait of a user, the method comprising:
receiving sensor data of the user captured by a multi-modal sensor array;
calculating a set of gait metrics, based on the sensor data captured during each sensor data window of a predetermined length, quantifying a gait of the user during the sensor data window;
encoding each set of gait metrics to form a motion embedding for the sensor data window;
inferring a patient state of the user, by a transformer-based network, based on the motion embedding generated for the sensor data window;
generating instructions or neurostimulations, by the transformer-based network, to induce the user to perform a physical movement;
receiving subsequent sensor data of the user performing the induced physical movement; and
using the subsequent sensor data as a dynamic input to induce a subsequent physical movement of the user.
